(19) United States
(12) Patent Application Publication
(10) Pub. No.: US 2011/0270017 A1
(43) Pub. Date: Nov. 3, 2011

(54) SELF-ADJUSTING GASTRIC BAND HAVING VARIOUS COMPLIANT COMPONENTS

(75) Inventors: Sean Snow, Goleta, CA (US); Babak Honaryar, Orinda, CA (US); Christian Y. Perron, Goleta, CA (US)

(73) Assignee: ALLERGAN, INC., Irvine, CA (US)

(21) Appl. No.: 12/770,617

(22) Filed: Apr. 29, 2010

(51) Int. Cl. A61F 2/04; A61B 17/12

(52) U.S. Cl. 600/37; 606/157

(57) ABSTRACT

A self-adjusting gastric band is automatically adjustable without complicated fluid control mechanisms, flow rate limiting devices, and/or valves. The self-adjusting gastric band may automatically adjust to allow a large bolus of food to pass through a constriction in the patient's stomach formed by the gastric band. The self-adjusting gastric band comprises an inflatable portion that is disposable about an esophageal-gastric junction of the patient. A first compliant portion is coupled to the inflatable portion. The first compliant portion automatically relaxes the constriction formed by the self-adjusting gastric band and allows the large bolus to pass through the constriction. After the bolus passes through the constriction, the self-adjusting gastric band automatically returns to its previous state.

### FIELD

The present invention generally relates to medical systems and apparatus and uses thereof for treating obesity and/or obesity-related diseases, and more specifically, relates to gastric banding systems that self-adjust to changes in a patient.

### BACKGROUND

Adjustable gastric banding apparatus have provided an effective and substantially less invasive alternative to gastric bypass surgery and other conventional surgical weight loss procedures. Despite the positive outcomes of invasive weight loss procedures, such as gastric bypass surgery, it has been recognized that sustained weight loss can be achieved through a laparoscopically-placed gastric band, for example, the LAP-BAND® (Allergan, Inc., Irvine, Calif.) gastric band or the LAP-BAND AP® (Allergan, Inc., Irvine, Calif.) gastric band. Generally, gastric bands are placed about the cardia, or upper portion, of a patient's stomach forming a stoma that restricts food's passage into a lower portion of the stomach. When the stoma is of an appropriate size that is restricted by a gastric band, food held in the upper portion of the stomach may provide a feeling of satiety or fullness that discourages overeating. Unlike gastric bypass procedures, gastric band apparatus are reversible and require no permanent modification to the gastrointestinal tract. An example of a gastric banding system is disclosed in Roslin, et al., U.S. Patent Pub. No. 2006/0235448, the entire disclosure of which is incorporated herein by this specific reference.

Over time, a stoma created by a gastric band may need adjustment in order to maintain an appropriate size, which is neither too restrictive nor too passive. Accordingly, prior art gastric band systems provide a subcutaneous fluid access port connected to an expandable or inflatable portion of the gastric band. By adding fluid to or removing fluid from the inflatable portion by means of a hypodermic needle inserted into the access port, the effective size of the gastric band can be adjusted to provide a tighter or looser constriction.

Sometimes, adjustment of a gastric band may be desirable in between adjustments made by a physician. For example, during normal operation of the gastric band, the band applies pressure to the outer surface of the upper stomach. But in some instances, the patient may swallow a bolus that is too large to pass through the constriction produced by the band. The result can be a painful experience which, if it persists, may require medical intervention to release the blockage.

Some attempts have been made to account for this possibility of blockage. For example, with reference to FIG. 1, Coe, et al., U.S. Patent Pub. No. 2009/0216255 discloses a flow control device A that moves fluid between a hydraulic restriction system and a fluid source B. The additional flow control device A controls a rate of fluid flow between the restriction device and the fluid source B. With reference to FIG. 2, Coe, et al., European Patent Application No. 2 074 970 A1 discloses a separate restriction device and pressure adjustment device C. The pressure adjustment device C regulates a constant force applied by the restriction device using, for example, a bellows and a spring.

With reference to FIG. 3, Lechner, U.S. Patent Pub. No. 2009/0054914 discloses a controllable stomach band that has a chamber for controlling restriction of the stomach band. The chamber is coupled to a separate pressure chamber D that receives fluid leaving the chamber in the stomach band. The pressure chamber D is separated from the esophageal-gastric junction of the patient's stomach.

Further, Steffen, U.S. Patent Pub. No. 2009/0062826 discloses an adjustable gastric band with a “conveyance device” that is powered by a “power storage device.” The power storage device operates the conveyance device to move fluid between expandable chambers to adjust the gastric band.

Accordingly, it is desirable to develop a self-adjusting gastric band that will provide the needed pressure to the stomach to create the stoma and facilitate weight control, but that will also adapt and open up to allow a large bolus to pass through. It is further desirable to create an automatically self-adjusting gastric band that does not require an electrical power source and/or external adjustments, to allow a large bolus to pass through.

Additionally, it is desirable to make the adjustments without additional, complicated fluid control mechanisms, flow rate limiting devices, and/or valves to regulate the transfer of fluid within the self-adjusting gastric band. Moreover, it is desirable to make these adjustments to the gastric band utilizing compliant components to both reduce and restore the constriction of the gastric band.

### SUMMARY

Generally described herein are automatic, self-adjusting, gastric banding systems that are capable of automatically relaxing and contracting in response to a large bolus passing through the area of a patient's stomach constricted by a gastric band. The apparatus and systems described herein aid in facilitating obesity control and/or treating obesity-related diseases while being non-invasive once implanted. Furthermore, the self-adjusting gastric banding systems disclosed herein are automatically adjustable without complicated fluid control mechanisms, flow rate limiting devices, and/or valves. The automatic adjustments may also be made in response to other changes in the patient's esophageal-gastric junction, for example, in response to size, shape, and or location changes.

In one embodiment, a self-adjusting gastric band automatically adjusts to allow a large bolus of food to pass through a constriction in the patient's stomach formed by the gastric band. The gastric band comprises an inflatable portion that is disposable about an esophageal-gastric junction of the patient. The gastric band also comprises an access port fluidly coupled to the inflatable portion via tubing to fill and drain the inflatable portion.

Further, the gastric band comprises a first compliant portion coupled to a part of the system. For example, the first compliant portion may be coupled to the inflatable portion, the access port, and/or the tubing. The first compliant portion automatically relaxes the constriction formed by the self-adjusting gastric band and allows the large bolus to pass through the constriction. After the bolus passes through the constriction, the gastric band automatically returns to its previous state.

In accordance with various embodiments, the first compliant portion facilitates automatically relaxing the constriction formed by the self-adjusting gastric band without causing a fluid to exit the inflatable portion of the gastric band. For example, the self-adjusting gastric band may comprise a ring coupled to the inflatable portion of the gastric band. The ring provides structure and support to the inflatable portion, and the ring facilitates disposing the inflatable portion about the esophageal-gastric junction.

The ring may be a flexible ring with a diameter that expands when a predetermined pressure is generated in the inflatable portion. For example, the predetermined pressure may be generated in response to the large bolus passing through the esophageal-gastric junction. The flexible ring expands to automatically relax the constriction formed by the self-adjusting gastric band. In various embodiments, the ring has a durometer in the range of approximately 20 to approximately 70.

According to a further embodiment, the first compliant portion receives a first amount of fluid from the inflatable portion when the large bolus causes a pressure in the first compliant portion to exceed an expansion pressure. Receiving the first amount of fluid from the inflatable portion facilitates relaxing the constriction formed by the self-adjusting gastric band and allowing the large bolus to pass through the constriction.

In an embodiment, the first compliant portion is fluidly coupled to the inflatable portion. The first compliant portion facilitates removing the first amount of fluid from the inflatable portion when the large bolus passes through the constriction.

According to another embodiment, the self-adjusting gastric band further comprises a second compliant portion fluidly coupled to the access port. The second compliant portion automatically removes a second amount of fluid from the inflatable portion via the access port to facilitate relaxing the constriction formed by the inflatable portion.

The tubing of the gastric banding system may be compliant tubing that expands in response to a pressure in the tubing exceeding a tubing expansion pressure when the large bolus passes through the constriction formed by the self-adjusting gastric band. In this regard, a third amount of fluid is removed from the inflatable portion when the compliant tubing expands. The tubing may be perforated to facilitate receiving the fluid from the inflatable portion via the tubing.

Further, another embodiment of the self-adjusting gastric band comprises a third compliant portion fluidly coupled to the tubing for automatically receiving a third amount of fluid from the inflatable portion via the tubing when the large bolus enters the esophageal-gastric junction. Receiving the third amount of fluid from the inflatable portion facilitates relaxing the constriction formed by the gastric band and allowing the large bolus to pass through the constriction.

The compliant components, according to various embodiments, comprise a kink-resisting feature. Further, the compliant components may comprise a leak-resisting feature. These components may be an elastic polymer, a balloon, a rubber container, a silicone container, a collapsible container, a bellows, and combinations thereof.

### BRIEF DESCRIPTION OF THE DRAWINGS

FIG. 1 illustrates a prior art system that includes a flow rate limiting device.

FIG. 2 illustrates a prior art system that includes a fluid control mechanism.

FIG. 3 illustrates a prior art system that includes a valve and a chamber separated from the esophageal-gastric junction.

FIG. 4 illustrates an exploded, perspective view of a self-adjusting gastric banding system according to an embodiment of the present invention.

FIG. 5 illustrates an exploded, perspective view of a self-adjusting gastric banding system having various compliant components according to an embodiment of the present invention.

FIG. 6 illustrates an exploded, perspective view of another self-adjusting gastric banding system having various compliant components according to an embodiment of the present invention.

FIG. 7 illustrates a chart showing pressure-volume curves for a standard gastric band and a self-adjusting gastric band according to an embodiment of the present invention.

FIG. 8 illustrates a chart showing pressure-time curves for a standard gastric band and a self-adjusting gastric band subject to a period of obstruction according to an embodiment of the present invention.

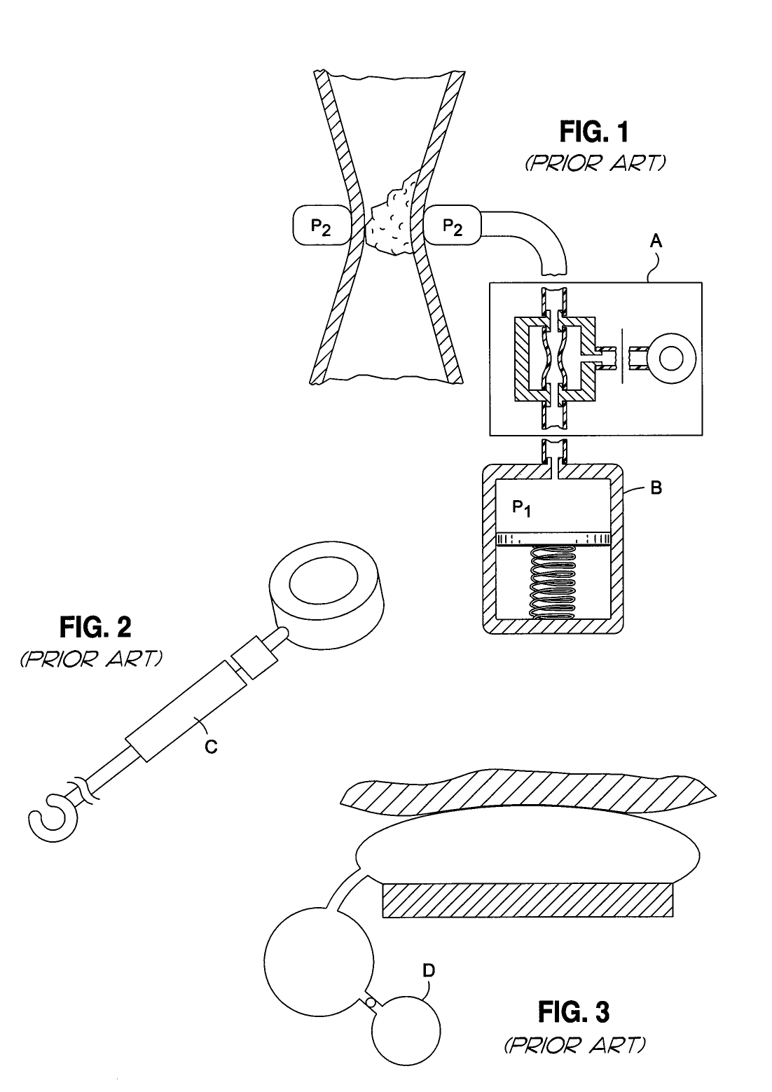

### DETAILED DESCRIPTION

The present invention generally provides self-adjusting gastric banding systems, for example, for treatment of obesity and obesity related conditions, as well as systems for allowing automatic self-adjustment of gastric bands when a patient swallows a large bolus.

Self-adjusting gastric bands are effective in helping a patient lose weight when the band is properly tightened around the patient's esophageal-gastric junction. During normal operation, the band applies pressure to the outer surface of the upper stomach. But, in some instances, the patient may swallow a bolus which is too large to pass through the constriction produced by the band—for example, when the patient swallows a large piece of steak. The result can be a painful experience which, if it persists, may require medical intervention to release the blockage.

In accordance with various embodiments of the present invention, the self-adjusting gastric band provides the needed pressure to the stomach to encourage weight loss. However, when a large bolus of food is swallowed, the self-adjusting gastric band temporarily and automatically opens up to allow the bolus through. After the bolus passes through, the mechanisms within the band return the band to its original size and shape. In an embodiment, electrical power and/or power external to the patient is not utilized to perform these adjustments. Further, in an embodiment, complicated fluid control mechanisms, flow rate limiting devices, and/or valves are not utilized to regulate the transfer of fluid within the self-adjusting gastric band.

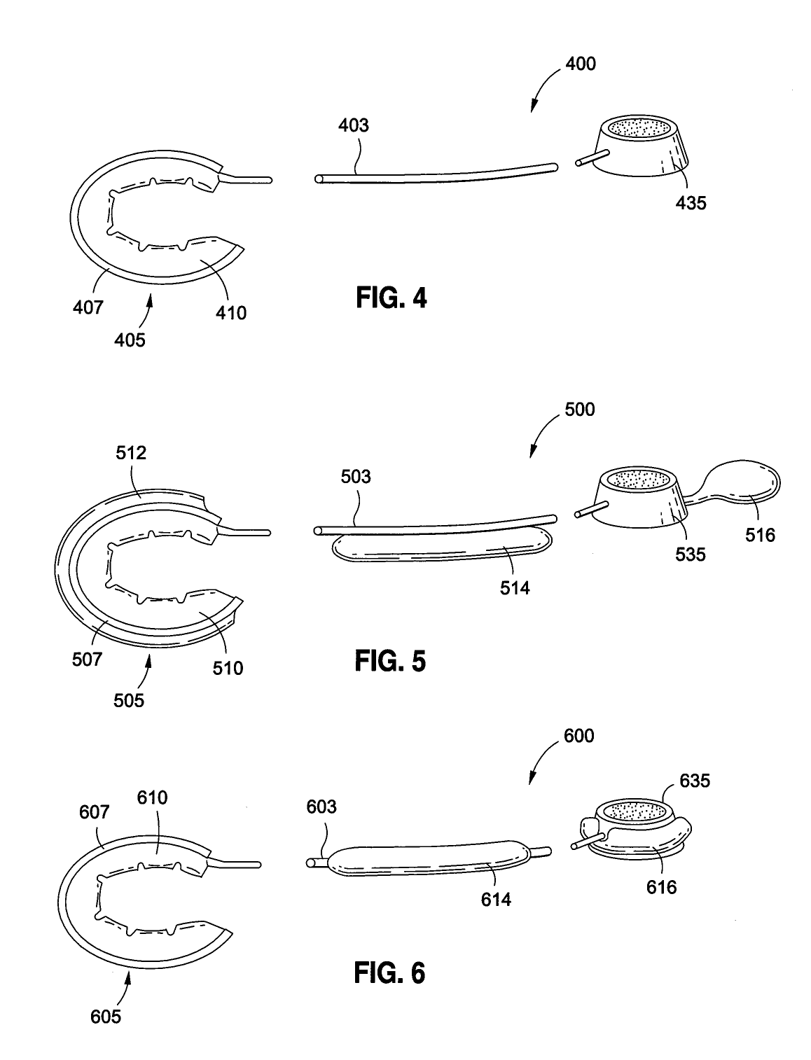

Turning now to FIG. 4, a self-adjusting gastric banding system 400 comprises a gastric band 405 coupled to a subcutaneous injection port 435 via tubing 403. The gastric band 405 comprises a circular ring 407 and an inflatable portion 410 disposed on the inside of the ring 407. The inflatable portion 410 separates the patient's stomach from the ring 407 when the gastric band 405 is implanted around the esophageal-gastric junction of the patient's stomach. The ring 407 provides structure and support to the inflatable portion 410, and facilitates implanting the gastric band 405 around the patient's stomach.

The access port 435 may be sutured onto the rectus muscle sheath or any other conveniently accessible muscle. The rectus muscle sheath provides a secure surface on which to attach the access port 435 under a layer of fat that separates the patient's skin from the muscle.

The inflatable portion 410 may be filled and drained with a fluid via the tubing 403. For example, the tubing 403 may be connected to the subcutaneous access port 435 for filling and draining the inflatable portion 410 via subcutaneous injections. The inflatable portion 410 may also be coupled to a reservoir to facilitate automatic adjustment of the inflatable portion 410, and the constriction it causes, when a large bolus attempts to pass through the constriction. When more fluid is introduced in the inflatable portion 410, the constriction around the stomach generally becomes tighter. Correspondingly, when less fluid is present, the constriction loosens and/or opens up.

The fluids used within the gastric band 405 may include any fluid that is biocompatible and incompressible. The fluid has no adverse effect on the patient in the unlikely event that a leak emanates from the system. The fluid can simply be water or any biocompatible polymer oil such as caster oil. In an example embodiment, the fluid is saline, a drug, and/or combinations thereof.

In an embodiment, the ring 407 is designed to be a compliant portion of the gastric band 405. For example, the ring 407 may flex and/or expand in response to a bolus of food moving through the constriction caused by the gastric band 405. The ring 407 may have flexible components and rigid components, such that the flexible components expand when a certain elevated and/or maximum pressure is reached in the inflatable portion 410. This elevated pressure may exist due to the presence of an obstruction such as a bolus near the gastric band 405. As the ring 407 expands, the diameters of the ring 407 and the inflatable portion 410 increase, and the constriction on the stomach due to the gastric band 405 is reduced to allow the bolus to pass through. When the bolus has passed, the elevated pressure no longer exists, and the gastric band 405 returns to the pre-obstruction state.

In another embodiment, the entire ring 407 may be flexible and/or expandable such that a diameter of the ring 407 increases in response to the elevated pressure in the inflatable portion 410. For example, the ring 407 may be constructed of silicone that has a durometer in the range of approximately 20 to approximately 70.

It should be understood that the flexible ring 407 and the other mechanisms disclosed herein for automatically adjusting the constriction of the gastric band 405 are only example embodiments. Any mechanism for automatically adjusting the constriction of the gastric band 405 that does not include electrical power, power external to the patient, complicated fluid control mechanisms, flow rate limiting devices, and/or valves is contemplated within the scope of the present invention.

Furthermore, although various compliant components are illustrated in each of the figures, it should be understood that any combination of the various compliant components may be utilized in different embodiments. For example, an embodiment may include one compliant component—only the ring, the tubing, or the access port may be compliant. In other embodiments, any combination of the ring, the tubing, and the access port may be compliant. For example, an embodiment may include a compliant ring and a compliant port, an embodiment may include compliant tubing and a compliant port, or an embodiment may include a compliant ring and compliant tubing. Any combination of compliant components is contemplated within the scope of the present invention.

With reference to FIG. 5, various compliant components may be utilized to automatically adjust the constriction of the gastric band 505 about the esophageal-gastric junction of the patient's stomach. Although three compliant components are illustrated in FIG. 5, as noted above, one or more of the components may be present in various embodiments of the present invention.

For example, in an embodiment, a band compliant component 512 is fluidly coupled to the inflatable portion 510 of the gastric band 505. The compliant component 512 is located on the outside of the ring 507, opposite the inflatable portion, and may be coupled to the ring 507 and the inflatable portion. Further, in an embodiment, one or more fluid ports may extend from the inflatable portion 510 to the compliant component 512 to fluidly couple the inflatable portion 510 to the compliant component 512.

With reference to FIGS. 5 and 6, and in accordance with various embodiments, a tube compliant component 514, 614 may be fluidly coupled to the tubing 503, 603. As illustrated in FIG. 6, the compliant component 614 may run along substantially the entire length of the tubing 603. In another embodiment, as illustrated in FIG. 5, the compliant component 514 may be limited to a smaller section of the entire length of the tubing 503. The compliant component 514, 614 may be fluidly coupled to the tubing 503 at one or more locations. For example, with reference to FIG. 6, the compliant component 614 and the tubing 603 may be perforated to allow for efficient transfer of the fluid between the tubing 603 and the compliant component 614.

In another embodiment, the tubing 603 itself may be compliant, and the durometer, thickness, and/or diameter of the tubing 603 may be altered to achieve a desired degree of compliance. Other components of the gastric band 605 may similarly have altered properties in order to achieve a desired degree of compliance.

In an embodiment where the tube compliant component 514, 614 facilitates automated adjustment of the gastric band 505, 605, the compliant component 514, 614 may have features configured to resist kinking and/or leakage of the tubing 503, 603. For example, the compliant component 514, 614 may include rigid portions (e.g., similar to a skeleton) and flexible portions. The rigid components may give structure to the compliant component 514, 614 and/or the tubing 503, 603 to prevent kinking and/or leakage due to external forces on the compliant component 514, 614 and/or the tubing 503, 603. The flexible components may automatically expand in response to an increased pressure in the inflatable portion 510, 610 of the gastric band 505, 605.

In accordance with another embodiment, and with continued reference to FIGS. 5 and 6, the access port 535, 635 may be fluidly coupled to a port compliant component 516, 616. As illustrated in FIG. 5, the compliant component 516 may be a balloon, reservoir, or other expandable device that is adjacent to the port 535. In an embodiment as illustrated in FIG. 6, the compliant component 616 may substantially surround the access port 635. The compliant component 616 may be fluidly coupled to the access port 635 at a single location near a coupling between the tubing 603 and the access port 635. In another embodiment, the compliant component 616 may be fluidly coupled to the access port 635 at multiple locations.

As noted above, any combination of the inflatable portion compliant component 512, a compliant ring 407 the tube compliant component 514, 614, and/or the port compliant component 516, 616 may be used in accordance with various embodiments. When the pressure in the inflatable portion 510, 610 exceeds a predetermined pressure, the compliant components 407, 512, 514, 516, 614, 616, in any particular configuration or combination, expand to receive an amount of the fluid from the inflatable portion 510, 610 via the inflatable portion 510, 610, the tubing 503, 603, and/or the access port 535, 635, and/or to reduce the constriction formed by the gastric band 405, 505, 605. The predetermined pressure may be predetermined based on a pressure that would indicate an obstruction is attempting to pass through the constriction caused by the gastric band 405, 505, 605.

The compliant components 407, 512, 514, 516, 614, 616 described herein, in accordance with various embodiments, may be designed with an expansion pressure at which pressure the components 407, 512, 514, 516, 614, 616 begin to expand, to receive fluid from the inflatable portion 510, 610 of the gastric band 505, 605, and/or to reduce the constriction formed by the gastric band 405, 505, 605. The expansion pressure may be configured to correspond to a predetermined pressure in the inflatable portion 410, 510, 610 that may indicate an obstruction exists in the esophageal-gastric junction.

For example, the obstruction may result in a large spike in intra-esophageal pressure that exceeds the expansion pressure and causes the compliant components to expand and receive fluid from the inflatable portion 510, 610. The reduction in fluid in the inflatable portion 510, 610 causes the constriction around the patient's stomach to loosen, in order to relieve the spike in pressure and allow the obstruction to pass through the esophageal-gastric junction. When the obstruction passes, the increased pressure in the inflatable portion 510, 610 is reduced, and the fluid flows back into the inflatable portion 510, 610 due to the elasticity of the compliant components 512, 514, 516, 614, 616, to restore the original amount of constriction of the gastric band 505, 605. This change in constriction of the gastric band 505, 605 results or is achieved without the use of flow rate limiting devices or valves.

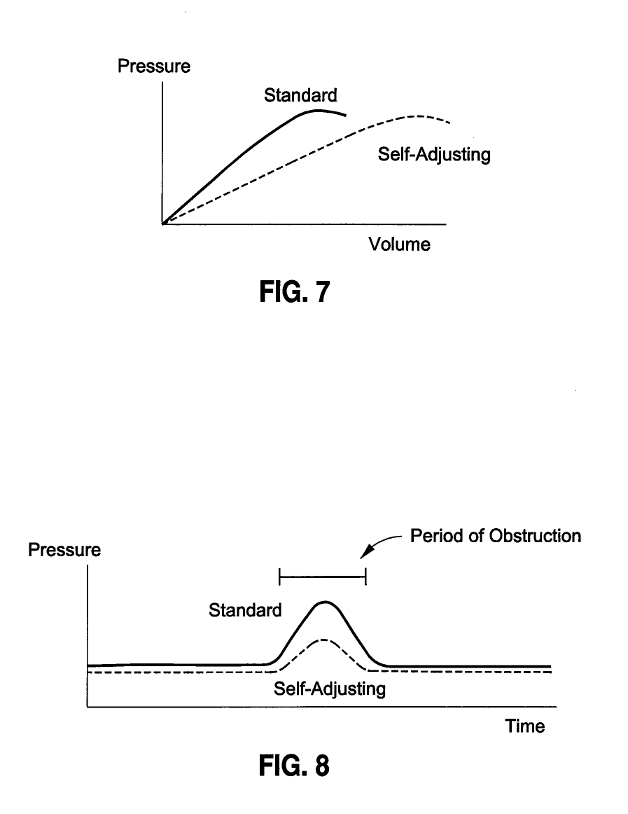

The graph in FIG. 7 illustrates, according to various embodiments, the effect the compliant components described herein have on the pressure in the gastric banding system. As can be seen in FIG. 7, a standard gastric banding system without compliant components has a certain pressure-volume relationship. After the gastric banding system is flushed with saline to remove any air trapped within the system (e.g., in the gastric band, the tubing, and the port), the pressure-volume relationship generally takes the form illustrated by the “Standard” curve in FIG. 7. The dashed “Compliant” curve illustrates an example embodiment of the pressure-volume relationship for a gastric banding system with one or more compliant components. As illustrated, the self-adjusting gastric banding system may include a greater volume of saline than a standard gastric banding system for a given level of pressure.

The graph in FIG. 8 illustrates, according to various embodiments, pressure characteristics of a “Standard” gastric banding system and a “Self-Adjusting” gastric banding system during use of the systems in a patient. Initially, the two systems are set to the same operating pressure, for example, for a desired level of constriction of the patient's stomach. As a large bolus of food or some other obstruction encounters the gastric band, the pressure in each system increases. As illustrated, the standard system has a larger pressure increase during the period of obstruction than the self-adjusting gastric banding system experiences. This smaller increase in pressure, according to various embodiments, is due to the addition of the reservoir space in the compliant component(s). As pressure in the gastric banding system increases, fluid is transferred into the reservoir space. Once the obstruction passes, the fluid is automatically returned from the reservoir space back into the gastric band.

The various compliant components disclosed herein may have any shape or configuration that facilitates removing an amount of fluid from the inflatable portion of the gastric band in response to an increased pressure in the inflatable portion. For example, the compliant components may be selected from a group consisting of a compressible reservoir, an elastic polymer, a balloon, a rubber container, a silicone container, a collapsible container, a bellows, and combinations thereof that are configured to contain the fluid.

Unless otherwise indicated, all numbers expressing quantities of ingredients, volumes of fluids, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, certain references have been made to patents and printed publications throughout this specification. Each of the above-cited references and printed publications are individually incorporated herein by reference in their entirety.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A self-adjusting gastric band that adjusts to allow a large bolus of food to pass through a constriction in a patient's stomach formed by the self-adjusting gastric band, the self-adjusting gastric band comprising:
an inflatable portion disposable about an esophageal-gastric junction of the patient;
an access port fluidly coupled to the inflatable portion via tubing to fill and drain the inflatable portion; and
a first compliant portion coupled to at least one of the inflatable portion, the access port, and the tubing, the first compliant portion for automatically relaxing the constriction formed by the self-adjusting gastric band and allowing the large bolus to pass through the constriction.

2. The self-adjusting gastric band of claim 1 further comprising a ring coupled to the inflatable portion for providing structure and support to the inflatable portion, wherein the ring facilitates disposing the inflatable portion about the esophageal-gastric junction.

3. The self-adjusting gastric band of claim 2 wherein the ring is a flexible ring having a diameter that expands when a predetermined pressure is generated in the inflatable portion in response to the large bolus passing through the esophageal-gastric junction, the flexible ring expanding to automatically relax the constriction formed by the self-adjusting gastric band.

4. The self-adjusting gastric band of claim 3 wherein the ring has a durometer in the range of approximately 20 to approximately 70.

5. The self-adjusting gastric band of claim 1 wherein the first compliant portion facilitates automatically relaxing the constriction formed by the self-adjusting gastric band without causing a fluid to exit the inflatable portion of the self-adjusting gastric band.

6. The self-adjusting gastric band of claim 1 wherein the first compliant portion receives a first amount of fluid from the inflatable portion when the large bolus causes a pressure in the first compliant portion to exceed an expansion pressure.

7. The self-adjusting gastric band of claim 6 wherein receiving the first amount of fluid from the inflatable portion facilitates relaxing the constriction formed by the self-adjusting gastric band and allowing the large bolus to pass through the constriction.

8. The self-adjusting gastric band of claim 1 wherein the first compliant portion is fluidly coupled to the inflatable portion, the first compliant portion receiving a first amount of fluid from the inflatable portion when the large bolus passes through the constriction.

9. The self-adjusting gastric band of claim 8 further comprising a second compliant portion fluidly coupled to the access port, the second compliant portion automatically receiving a second amount of fluid from the inflatable portion via the access port to facilitate relaxing the constriction formed by the inflatable portion.

10. The self-adjusting gastric band of claim 9 wherein the tubing is a compliant tubing that expands in response to a pressure in the tubing exceeding a tubing expansion pressure when the large bolus passes through the constriction formed by the self-adjusting gastric band.

11. The self-adjusting gastric band of claim 10 wherein a third amount of fluid is removed from the inflatable portion when the compliant tubing expands.

12. The self-adjusting gastric band of claim 9 further comprising a third compliant portion fluidly coupled to the tubing for automatically receiving a third amount of fluid from the inflatable portion via the tubing when the large bolus enters the esophageal-gastric junction.

13. The self-adjusting gastric band of claim 12 wherein receiving the third amount of fluid from the inflatable portion facilitates relaxing the constriction formed by the self-adjusting gastric band and allowing the large bolus to pass through the constriction.

14. The self-adjusting gastric band of claim 12 wherein the third compliant portion extends along a length of the tubing.

15. The self-adjusting gastric band of claim 14 wherein the third compliant portion is perforated to facilitate receiving the third amount of fluid via the tubing.

16. The self-adjusting gastric band of claim 12 wherein at least one of the first compliant portion, the second compliant portion, and the third compliant portion comprise a kink-resisting feature.

17. The self-adjusting gastric band of claim 12 wherein at least one of the first compliant portion, the second compliant portion, and the third compliant portion comprises a leak-resisting feature.

18. The self-adjusting gastric band of claim 12 wherein the first compliant portion, the second compliant portion, and the third compliant portion are selected from a group consisting of an elastic polymer, a balloon, a rubber container, a silicone container, a collapsible container, a bellows, and combinations thereof.

19. A self-adjusting gastric band that adjusts to allow a large bolus of food to pass through a constriction in a patient's stomach formed by the self-adjusting gastric band, the self-adjusting gastric band comprising:
an inflatable portion wherein an amount of fluid is removed from the inflatable portion to automatically relax the constriction formed by the self-adjusting gastric band and allow the large bolus to pass through the constriction;
a ring coupled to the inflatable portion to facilitate disposing the self-adjusting gastric band about an esophageal-gastric junction of the patient; and
a compliant portion fluidly coupled to the inflatable portion, the compliant portion for receiving the amount of fluid from the inflatable portion without a valve or a flow rate limiting device.

20. A self-adjusting gastric banding system that adjusts a gastric band when a large bolus of food is swallowed by a patient, to allow the large bolus to pass through a constriction in a patient's stomach formed by the gastric band, the system comprising:
an inflatable portion of the gastric band, the gastric band being disposable about an esophageal-gastric junction of the patient;
a ring coupled to the inflatable portion for providing support to the inflatable portion and for securing the inflatable portion around the patient's stomach, wherein the ring flexes in response to the large bolus to facilitate allowing the large bolus to pass through the constriction formed by the gastric band;
a first compliant portion fluidly coupled to the inflatable portion for automatically receiving a first amount of fluid from the inflatable portion when the large bolus enters the esophageal-gastric junction, to facilitate automatically relaxing the constriction formed by the gastric band and allowing the large bolus to pass through the constriction;
an access port fluidly coupled to the inflatable portion via tubing to fill and drain the inflatable portion;
a second compliant portion fluidly coupled to the access port for automatically receiving a second amount of fluid from the access port when the large bolus enters the esophageal-gastric junction, to facilitate automatically relaxing the constriction formed by the gastric band and allowing the large bolus to pass through the constriction; and
a third compliant portion fluidly coupled to the tubing for automatically receiving a third amount of fluid from the tubing when the large bolus enters the esophageal-gastric junction, to facilitate automatically relaxing the constriction formed by the gastric band and allowing the large bolus to pass through the constriction.
